(19) United States
(12) Patent Application Publication
(10) Pub. No.: US 2014/0235688 A1
(43) Pub. Date: Aug. 21, 2014

(54) TREATMENT OF EXTERNAL PARASITES BY ORAL ADMINISTRATION OF PARASITICIDAL AGENTS

(71) Applicant: Piedmont Pharmaceuticals, LLC., Greensboro, NC (US)

(72) Inventors: Roland H. Johnson, Lexington, NC (US); Douglas I. Hepler, McLeansville, NC (US); Kathleen G. Palma, McLeansville, NC (US)

(73) Assignee: Piedmont Pharmaceuticals, LLC., Greensboro, NC (US)

(21) Appl. No.: 14/046,820

(22) Filed: Oct. 4, 2013

Related U.S. Application Data
(63) Continuation-in-part of application No. 12/471,129, filed on May 22, 2009.
(60) Provisional application No. 61/102,774, filed on Oct. 3, 2008.

(51) Int. Cl. A61K 9/00; A61K 45/06; A61K 31/415

(52) U.S. Cl. CPC A61K 9/0053; A61K 31/415; A61K 45/06
USPC 514/404; 548/367.4

(57) ABSTRACT

Pharmaceutically acceptable compositions of fipronil for oral delivery to mammals and methods for use of the compositions to control external parasites, such as fleas and ticks.

### RELATED APPLICATIONS

This Application is a continuation-in-part of U.S. patent application Ser. No. 12/471,129, filed on May 22, 2009, which in turn claims the benefit of the priority of U.S. Provisional Patent Application Ser. No. 61/102,774, filed on Oct. 3, 2008. The disclosures of these applications is incorporated herein by this reference.

### FIELD OF THE INVENTION

The invention relates to the oral administration of ectoparasiticidal agents. More particularly, the invention relates to administration of a phenylpyrazole (such as fipronil) or its derivatives to mammals to treat external parasites.

### BACKGROUND OF THE INVENTION

With the exception of lufenuron, an insect growth regulator, the market for ectoparasite control in mammals has long been dominated by compositions for topical administration. The latter includes the two top-selling products for flea and tick control, FRONTLINE® (fipronil) with efficacy against fleas, ticks and scabies sold for use on dogs and cats, and ADVANTAGE® (imidacloprid) with efficacy against fleas (but not ticks), also sold for use on dogs and cats. Both active compounds can be toxic at relatively low oral dosages. Fipronil in particular is known to cause emesis when given orally in relatively high concentrations for daily doses based on toxicity studies conducted to support EPA approval of fipronil for topical use (in view of the possibility that a treated animal could ingest small quantities of the drug while grooming). No systemic effect on parasites from oral administration was intended or shown. Instead, the topical product (FRONTLINE®) is believed to act by binding of the active to lipids in hair follicles, where it remains for extended periods of time.

Topical routes of administration raise concerns regarding the administered compound's potentially toxic effect on humans. It is generally advised that humans not contact the administration site for several hours after application. Compliance with this advice as to treatment of domestic pets can be difficult, especially for children. In addition, the carriers used for such topical products often contain waxes, oils or other spreading agents, and so remain on the treated animal's skin for a length of time sufficient to risk environmental contamination or staining. Furthermore, certain products containing these actives are used in combination with other agents that are toxic to other species, such as permethrin, which is suitable for use in dogs but is potentially lethal to cats, making topical use of such products problematic in multi-species environments.

It is therefore desirable to provide orally deliverable fipronil and a non-toxic regimen for its administration with efficacy in killing of, and prevention of reinfestations by, external parasites, notably fleas and ticks.

### SUMMARY OF THE INVENTION

One embodiment of the invention is based on the surprising discovery that fipronil, when given orally at sub-toxic concentrations (as determined from dose concentrations at administration daily, weekly or monthly, with no more often than weekly or monthly being preferred) kills external parasites quickly, comprehensively and over extended durations. In particular, the effect is surprisingly long-lasting, extending up to 4 or more weeks after treatment, providing both a killing effect with respect to any existing infestation and a preventative effect with respect to reinfestation.

The invention therefore provides pharmaceutically acceptable compositions of fipronil for oral delivery to mammals to control external parasites. In another embodiment of this aspect of the invention, the external parasites are adult fleas or ticks. In a further embodiment of the invention, the external parasites are flea or tick eggs.

In a further embodiment of this aspect of the invention, the ectoparasiticidal agent present in the pharmaceutical composition is fipronil, without derivation or modification of the compound. In another aspect of this embodiment, this fipronil active is the only ectoparasiticidal agent present in the pharmaceutical composition.

### DESCRIPTION OF THE INVENTION

A. Active Ingredients of the Pharmaceutically Acceptable Compositions of the Invention.

By “pharmaceutically acceptable composition” it is meant that the active ectoparasiticidal agent present is formulated for oral delivery in a manner rendering the composition product acceptable for administration to humans or animals.

An active whose use is contemplated by the invention is fipronil, or its desulfinyl, sulfinyl, sulfide, or sulfone metabolites. The chemical structure of fipronil follows:

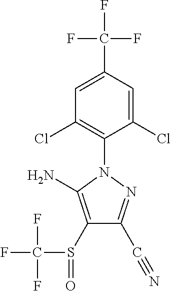 [SMILES: N#Cc1nn(-c2c(Cl)cc(C(F)(F)F)cc2Cl)c(N)c1S(=O)C(F)(F)F]

Fipronil: 5-amino-1-[2,6-dichloro-4-(trifluoromethyl)phenyl]-4-[(trifluoromethyl)sulfinyl]-1H-pyrazole-3-carbonitrile. As noted, certain metabolites of fipronil may also be useful in the invention. However, in a preferred embodiment, it is not necessary to modify the active compound itself to avoid adverse effects on the treated host, such as emesis.

Also, in preferred embodiments it is not necessary to include other active agents in the pharmaceutically acceptable compositions to achieve the desired level of efficacy. However, those of skill in the art will recognize that other active agents may have desirable properties, and so might be combined with or substituted for fipronil in the compositions of the invention for particular purposes, such as providing initial control of parasites in less than 24 hours or to kill eggs or larvae in additional to adult parasites. Examples of suitable actives for co-administration with the fipronil of the invention include lufenuron, imidacloprid, dinotfuran, pyriproxyfen, permethrin, metaflumazone, pyriprole, nitenpyram, S-methoprene and selamectin.

B. Ectoparasite Targets

The active compounds are suitable for the control of ectoparasites which can be found in humans and in animal keeping and livestock breeding in domestic animals, productive livestock, zoo animals, laboratory animals, experimental animals and pets, while having favorable toxicity to mammals at the dosages provided by the invention. The productive livestock and breeding animals include mammals such as, for example, cattle, horses, sheep, pigs, goats, camels, water buffalo, donkeys, rabbits, fallow deer, reindeer, fur-bearing animals such as, for example, mink, chinchilla, raccoon, birds such as, for example, chickens, geese, turkeys and ducks. Laboratory and experimental animals include mice, rats, guinea pigs, golden hamsters, dogs and cats. Pets include dogs and cats.

The pharmaceutical composition is ectoparasiticidally active against all developmental stages of the ectoparasites, including eggs and larvae. Targeted ectoparasites are those which bite the host or otherwise gain access to its blood during infestation, and include the order Siphonaptera (fleas) extending, for example, to the genera Ctenocephalides., Echidnophaga, Pulex and Ceratophyllus; the order Ixodida (ticks) including all infraorders, with emphasis on the family Ixodidae extending, for example, to the genera Ixodes, Dermacentor, Rhipicephalus, Amblyomma, Haemaphysalis and Boophilus as well as the family Argasidae extending, for example, to the genera Argasinae, Ornithodorinae, Otobinae, Antricolinae and Nothoaspinae; the order Anoplura (blood-sucking lice) extending, for example, to the genera Haematopinus, Linognathus, Solenopotes, Pediculus and Pthirus; the order Mallophaga (biting lice) extending, for example, to the genera Trimenopon, Menopon, Eomenacanthus, Menacanthus, Trichodectes, Felicola, Damalinea and Bovicola; the order Diptera extending, for example, to the biting species, such as those of the genus Ceratopogonidae; and from the order Astigmata, extending, for example, to the genus Sarcoptes (in the presence of blood).

C. Dosage Forms for the Pharmaceutical Compositions of the Invention.

1. Dosage Ranges.

Administration can be effected prophylactically as well as therapeutically. Effective dosage ranges of the pharmaceutical formulations of the fipronil composition for oral administration are in the range of 0.5 to 10 mg/kg, or 1 to 10 mg/kg, or 1 to 10 mg/kg, or about 1 to about 4 mg/kg, optionally 1 to 2 mg/kg, and also optionally 4 mg/kg and preferably 2 mg/kg. Dosing is preferably no more often than weekly, and most often no more often than monthly as necessary to achieve efficacy in the animal without toxicity.

Following oral administration of the pharmaceutical compositions of the present invention, the active agent passes though the mucosal barriers of the GI tract and is absorbed into the blood stream where it can be detected in the plasma of subjects. The level of active agent in the bloodstream as measured in the plasma is dose-dependent. The active agent may be co-administered with another active (e.g., to expedite the killing effect or target eggs and larvae), either in the same dosage form, or simultaneously therewith, or sequentially.

3. Dosage Vehicles.

The ectoparasiticidal composition can be provided in any therapeutically acceptable pharmaceutical form suitable for oral administration. The pharmaceutical composition can be formulated for oral administration as, for example but not limited to, drug powders, crystals, granules, small particles (which include particles sized on the order of micrometers, such as microspheres and microcapsules), particles (which include particles sized on the order of millimeters), beads, microbeads, pellets, pills, microtablets, compressed tablets or tablet triturates, molded tablets or tablet triturates, and in capsules, which are either hard or soft and contain the composition as a powder, particle, bead, solution or suspension. The pharmaceutical composition can also be formulated for oral administration as a solution or suspension in an aqueous liquid, as a liquid incorporated into a gel capsule or as any other convenient formulation for administration, or for rectal administration, as a suppository, enema or other convenient form. The ectoparasiticidal composition can also be provided as a controlled release system (see, e.g., Langer, 1990, Science 249: 1527-1533).

As to oral dosage forms of the present invention that are solid, the active may simply be provided in gelatin capsules, with or without optional pharmaceutical excipients. Suitable pharmaceutical excipients are known to those of ordinary skill in the art and include, in addition to those mentioned with respect to the chewable treat dosage form, the following: acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, tartaric acid); aerosol propellants (butane, dichlorodifluoro-methane, dichlorotetrafluoroethane, isobutane, propane, trichloromonofluoromethane); Air displacements (carbon dioxide, nitrogen); alcohol denaturants (denatonium benzoate, methyl isobutyl ketone, sucrose octacetate); alkalizing agents (strong ammonia solution, ammonium carbonate, diethanolamine, diisopropanolamine, potassium hydroxide, sodium bicarbonate, sodium borate, sodium carbonate, sodium hydroxide, trolamine); anticaking agents, such as glidants; antifoaming agents (dimethicone, simethicone); antimicrobial preservatives (benzalkonium chloride, benzalkonium chloride solution, benzelthonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, sorbic acid, thimerosal, thymol); antioxidants (ascorbic acid, acorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate); capsule lubricants (see tablet and capsule lubricant); chelating agents (edetate disodium, ethylenediaminetetraacetic acid and salts, edetic acid); coating agents (sodium carboxymethyl-cellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyethylene glycol, polyvinyl acetate phthalate, shellac, sucrose, titanium dioxide, carnauba wax, microcystalline wax, zein); colorants (caramel, red, yellow, black or blends, ferric oxide); complexing agents (ethylenediaminetetraacetic acid and salts (EDTA), edetic acid, gentisic acid ethanolmaide, oxyquinoline sulfate); desiccants (calcium chloride, calcium sulfate, silicon dioxide); emulsifying and/or solubilizing agents (acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 caster oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, soritan monooleate, sorbitan monopalmitate, sorbitan monostearate, stearic acid, trolamine, emulsifying wax); filtering aids (powdered cellulose, purified siliceous earth); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin); glidants and/or anticaking agents (calcium silicate, magnesium silicate, colloidal silicon dioxide, talc); humectants (glycerin, hexylene glycol, propylene glycol, sorbitol); plasticizers (castor oil, diacetylated monoglycerides, diethyl phthalate, glycerin, mono- and di-acetylated monoglycerides, polyethylene glycol, propylene glycol, triacetin, triethyl citrate); polymers (e.g., cellulose acetate, alkyl celloloses, hydroxyalkylcelloloses, acrylic polymers and copolymers); solvents (acetone, alcohol, diluted alcohol, amylene hydrate, benzyl benzoate, butyl alcohol, carbon tetrachloride, chloroform, corn oil, cottonseed oil, ethyl acetate, glycerin, hexylene glycol, isopropyl alcohol, methyl alcohol, methylene chloride, methyl isobutyl ketone, mineral oil, peanut oil, polyethylene glycol, propylene carbonate, propylene glycol, sesame oil, water for injection, sterile water for injection, sterile water for irrigation, purified water); sorbents (powdered cellulose, charcoal, purified siliceous earth); carbon dioxide sorbents (barium hydroxide lime, soda lime); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax); suspending and/or viscosity-increasing agents (acacia, agar, alginic acid, aluminum ‘ monostearate, bentonite, purified bentonite, magma bentonite, carbomer 934p, carboxymethylcellulose calcium, carboxymethylcellulose sodium, carboxymethycellulose sodium 12, carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, propylene glycol alginate, silicon dioxide, colloidal silicon dioxide, sodium alginate, tragacanth, xanthan gum); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup); tablet binders (acacia, alginic acid, sodium carboxymethylcellulose, microcrystalline cellulose, dextrin, ethylcellulose, gelatin, liquid glucose, guar gum, hydroxypropyl methylcellulose, methycellulose, polyethylene oxide, povidone, pregelatinized starch, syrup); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar); tablet disintegrants (alginic acid, microcrystalline cellulose, croscarmellose sodium, corspovidone, polacrilin potassium, sodium starch glycolate, starch, pregelatinized starch); gablet and/or capsule lubricants (calcium stearate, glyceryl behenate, magnesium stearate, light mineral oil, polyethylene glycol, sodium stearyl fumarate, stearic acid, purified stearic acid, talc, hydrogenated vegetable oil, zinc stearate); tonicity agents (dextrose, glycerin, mannitol, potassium chloride, sodium chloride); flavoring vehicles, including flavored and/or sweetened fluids (aromatic elixir, compound benzaldehyde elixir, iso-alcoholic elixir, peppermint water, sorbitol solution, syrup, tolu balsam syrup); oil vehicles, (almond oil, corn oil, cottonseed oil, ethyl oleate, isopropyl myristate, isopropyl palmitate, mineral oil, light mineral oil, myristyl alcohol, octyldodecanol, olive oil, peanut oil, persic oil, sesame oil, soybean oil, squalane); carrier vehicles (sugar spheres); viscosity-increasing agents (see suspending agent); water repelling agents (cyclomethicone, dimethicone, simethicone); and wetting and/or solubilizing agents (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaureate, sorbitan monooleate, sorbitan monopalmitate, sorbitan monostearate, tyloxapol). This list is not meant to be exclusive, but instead merely representative of the classes of excipients and the particular excipients which may be used in any oral dosage forms of the present invention.

In addition, a directly-compressible. ectoparasiticidal composition (i.e, that can be directly compressed, without excipients, into a tablet of pharmaceutically acceptable hardness and friability) compressed into a tablet, optionally with a lubricant, such as but not limited to magnesium stearate, and enteric coated. In another embodiment, the pharmaceutical compositions containing the ectoparasiticidal composition alternatively include one or more substances that either neutralize stomach acid and/or enzymes or are active to prevent secretion of stomach acid. These formulations can be prepared by methods known in the art, see, e.g., methods described in Remington's Pharmaceutical Sciences, 18th Ed., ed. Alfonso R. Getman), Mack Publishing Co., Easton, Pa., 1990.

The pharmaceutical compositions of the invention may include an enteric coating along with, optionally, another pharmaceutically acceptable vehicle. Enteric coatings are those coatings that remain intact in the stomach, but will dissolve and release the contents of the dosage form once it reaches the small intestine. A large number of enteric coatings are prepared with ingredients that have acidic groups such that, at the very low pH present in the stomach, i.e. pH 1.5 to 2.5, the acidic groups are not ionized and the coating remains in an undissociated, insoluble form. At higher pH levels, such as in the environment of the intestine, the enteric coating is converted to an ionized form, which can be dissolved to release the ectoparasiticidal composition. Other enteric coatings remain intact until they are degraded by enzymes in the small intestine, and others break apart after a defined exposure to moisture, such that the coatings remain intact until after passage into the small intestines.

Polymers which are useful for the preparation of enteric coatings include, but are not limited to, shellac, starch and amylose acetate phthalates, styrine-maleic acid copolymers, cellulose acetate succinate, cellulose acetate phthalate (CAP), polyvinylacetate phthalate (PVAP), hydroxypropylmethylcellulose phthalate (grades HP-50 and HP-55), ethylcellulose, fats, butyl stearate, and methacrylic acid-methacrylic acid ester copolymers with acid ionizable groups (“EUDRAGIT™.”), such as “EUDRAGIT™ L 30D”, “EUDRAGIT™. RL 30D”, “EUDRAGIT™ RS 30D”, “EUDRAGIT™. L 100-55”, and “EUDRAGIT™. L 30D-55”.

The disintegration of the enteric coating occurs either by hydrolysis by intestinal enzymes or by emulsification and dispersion by bile salts, depending upon the type of coating used. For example, esterases hydrolyze esterbutyl stearate to butanol and stearic acid and, as the butanol dissolves, the stearic acid flakes off of the medicament. Additionally, bile salts emulsify and disperse ethylcellulose, hydroxypropylmethylcellulose, fats and fatty derivatives. Other types of coatings are removed depending on the time of contact with moisture, for example coatings prepared from powdered carnauba wax, stearic acid, and vegetable fibers of agar and elm bark rupture after the vegetable fibers absorb moisture and swell. The time required for disintegration depends upon the thickness of the coating and the ratio of vegetable fibers to wax.

A coating may also be provided to help protect the stability of the active and mask its taste. To that end, food grade coatings are preferred, such as an aqueous film coat from Colorcon Corporation sold as OPADRY™. OPADRY is a methylcellulose based product with a plasticizer and pigment. Since the coating is aqueous based, no special handling precautions are required during manufacture. However, after administration, the aqueous film coat will start to erode and/or dissolve within minutes when exposed to water or other liquids in the stomach.

Application of a coating to the ectoparasiticidal composition can be accomplished by any method known in the art for applying enteric coatings. For example, but not by way of limitation, enteric polymers can be applied using organic solvent based solutions containing from 5 to 10% w/w polymer for spray applications and up to 30% w/w polymer for pan coatings. Solvents that are commonly in use include, but are not limited to, acetone, acetone/ethyl acetate mixtures, methylene chloride/methanol mixtures, and tertiary mixtures containing these solvents. Some enteric polymers, such as methacrylic acid-methacrylic acid ester copolymers can be applied using water as a dispersant. The volatility of the solvent system must be tailored to prevent sticking due to tackiness and to prevent high porosity of the coating due to premature spray drying or precipitation of the polymer as the solvent evaporates.

Furthermore, plasticizers can be added to a coating to prevent cracking of the coating film. Suitable plasticizers include the low molecular weight phthalate esters, such as diethyl phthalate, acetylated monoglycerides, triethyl citrate, polyethyl glycoltributyl citrate and triacetin. Generally, plasticizers are added at a concentration of 10% by weight of enteric coating polymer weight. Other additives such as emulsifiers, for example detergents and simethicone, and powders, for example talc, may be added to the coating to improve the strength and smoothness of the coating. Additionally, pigments may be added to the coating to add color to the pharmaceutical formulation.

In general, the ectoparasiticidal composition can also be prepared in granulated or powder form (e.g., for use in a feed-through dosing regimen) using any method known in the art, such as but not limited to, crystallization, spray-drying or any method of comminution, preferably using a high speed mixer/granulator. Examples of high speed mixer/granulators include the “LITTLEFORD LODIGE™.” mixer, the “LITTLEFORD LODIGE™” MGT mixer/granulator, and the “GRAL™.” mixer/granulator. During the high-shear powder mixing, solutions of granulating agents, called binders, are sprayed onto the powder to cause the powder particles to agglomerate, thus forming larger particles or granules. Granulating agents which are useful for preparing the ectoparasiticidal composition granules, include but are not limited to, cellulose derivatives (including carboxymethylcellulose, methylcellulose, and ethylcellulose), gelatin, glucose, polyvinylpyrrolidone (PVP), starch paste, sorbitol, sucrose, dextrose, molasses, lactose, acacia gum, sodium alginate, extract of Irish moss, panwar gum, ghatti gum, mucilage of isapol husks, Veegum and larch arabogalactan, polyethylene glycol, and waxes. Granulating agents may be added in concentrations ranging from 1 to 30% of the mass of the particles or granules. The ectoparasiticidal composition powder or granules may be coated; e.g., using standard fluidized bed equipment.

Ectoparasiticidal composition granules or powder particles can also be suspended in a solution for oral administration as a liquid. The suspension can be prepared from aqueous solutions to which thickeners and protective colloids are added to increase the viscosity of the solution to prevent rapid sedimentation of the coated powder particles or granules. Any material which increases the strength of the hydration layer formed around suspended particles through molecular interactions and which is pharmaceutically compatible with the ectoparasiticidal composition can be used as a thickener, such as but not limited to, gelatin, natural gums (e.g., tragacanth, xanthan, guar, acacia, panwar, ghatti, etc.), and cellulose derivatives (e.g., sodium carboxymethylcellulose, hydroxypropylcellulose, and hydroxypropylmethylcellulose, etc.). Optionally, a surfactant such as Tween may be added to improve the action of the thickening agent.

In another embodiment, the ectoparasiticidal composition is administered with a substance that inactivates or inhibits the action of stomach enzymes, such as pepsin. Alternatively, the pharmaceutical composition containing the ectoparasiticidal composition is administered either concurrent with or subsequent to administration of a pharmaceutical composition active to inactivate or inhibit the action of stomach enzymes. For example, but not by way of limitation, protease inhibitors, such as aprotin, can be used to inactivate stomach enzymes. In another embodiment, the ectoparasiticidal composition is formulated or administered with a compound or compounds which inhibit the secretion of stomach acid. Compounds which are useful for inhibiting the secretion of stomach acid include, but are not limited to, ranitidine, nizatidine, famotidine, cimetidine, and misoprostol.

Another example of a suitable dosage format for oral delivery of the pharmaceutical compositions of the invention is a chewable treat for edible consumption by the treated host. An example of a process for manufacturing such treats is one in which production of the treat is performed without generation of heat at a level that would cause the active to wholly or partially degrade. The method is preferably performed so the chew mixture and formed treats are not exposed to temperatures at or above those typically generated by compression and/or shear stress exerted in extrusion (e.g., the mixture is maintained at temperatures no more than about 10° above room temperature (20° C.) or 10° below room temperature). Stability of the actives is therefore preserved. A detailed description of a chewable formulation produced by a particular method for their manufacture are provided in co-pending and commonly owned U.S. patent application Ser. No. 12/471,129, filed on May 22, 2009, whose disclosure has been incorporated herein.

To improve palatability of oral compositions, flavorings are preferably present which are at least food grade in quality, and most preferably exclude animal origin flavorings. Preferred non-animal origin flavorings are plant proteins, such as soy protein, to which edible artificial food-like flavorings has been added (e.g., soy-derived bacon flavoring). Depending on the target animal, other non-animal flavorings could include anise oil, carob, peanuts, fruit flavors, sweeteners such as honey, sugar, maple syrup and fructose, herbs such as parsley, celery leaves, peppermint, spearmint, garlic, or combinations thereof.

A particularly preferred flavoring is Provesta™ 356, made by Ohly, Inc. It is a light tan, water-soluble powder that builds on the properties of yeast extracts and reaction flavors to provide a pleasant smoky, cured bacon flavor. Provesta 356 contains no animal derived ingredients.

For administration to horses and other grazing animals, as well as small animals such as rabbits, hamsters, gerbils, and guinea pigs, grains and seeds are especially appealing additional flavoring agents. The grains may be present in any form consistent with the production of the chew including flour, bran, cereal, fiber, whole grain and meal forms, including gluten meals, and may be rolled, crimped, ground, dehydrated or milled. Minerals may also be added as flavorings, such as salt and other spices. Preferably, the grain utilized is dehydrated, milled or flaked. Vegetables such as dehydrated carrots and seeds such as safflower seeds or milo seeds are especially appealing to small animals and may be included.

The invention having been fully described, its practice is illustrated (but not limited) by the following examples. Standard abbreviations and measurements apply throughout the examples unless a contrary definition is given.

### EXAMPLE I

### Oral Administration of Fipronil and Efficacy of Different Dosages Against Fleas and Ticks

On Day 0, the dogs were placed into 3′×4′ stainless steel cages with pans lined with white paper for ˜6 hrs post dose. Except for control animals, the dogs were infested either with ˜50 R. sanguineus ticks or ˜100 fleas. The paper liners were observed for evidence of fleas falling from the animals at approximately 30 minutes, one hour and 24 hours post dose. Blood was collected from the dogs at approximately 4 hours post-dose on Days 0 and 2. The blood was collected into lavender top Vacutainer® tubes (Lot #7278370, Exp. February 2009). The tubes were centrifuged and plasma was drawn off and frozen at approximately −20° C. pending possible future shipment to the sponsor for analysis of levels of test article in the plasma. Comb counts were conducted as indicated in Tables 1-9 below. The number of live fleas and ticks removed during the comb counts was recorded.

Results

Dosing information and results are presented in Tables 1-9, below.

Dosing concentrations were 1 mg/kg on day 0; 2 mg/kg on day 14; and 4 mg/kg on day 36. All doses were highly efficacious against fleas, with somewhat lesser efficacy against ticks. However, and unexpectedly, the 4 mg/kg dose provided excellent efficacy against both fleas and ticks over a duration of at least 4 weeks, potentially as long as 6 to 8 weeks. With an enteric coating and/or a controlled release dosing format, the same level of efficacy can be expected to be obtained with a 2 mg/kg dose concentration, with dosing no more often than weekly being possible, no more than monthly being preferred, and dosing no more often than 6 to 8 or 9 weeks being sufficient. At 12 weeks, however, efficacy for fleas declines to about 3.5% at 4 mg/kg.

Therefore, it is established for what is believed to be the first time that about two month cycle of oral dosing for fleas at 4 mg/kg is achievable according to the invention.

TABLE 1
Treated animals dosed at 1 mg/kg at Day 0.
Fipronil dosed at 1 mg/kg

| | | | Day 0 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 65 | |
| 4882 | 2 | fipronil | 0 | 0 | 17 | |
| 4871 | 2 | fipronil | 1 | 1 | 29 | |
| 4864 | 2 | fipronil | 0 | 0 | 11 | |
| | | | | Group 2 | 19.0 | 70.8% |
| | | | | mean: | | |
| 4857 | 3 | untreated | 0 | 0 | 22 | |
| 4897 | 4 | fipronil | 0 | 0 | 24 | |
| 4881 | 4 | fipronil | 0 | 0 | 14 | |
| 4853 | 4 | fipronil | 0 | 0 | 18 | |
| | | | | Group 4 | 18.7 | 15.2% |
| | | | | mean: | | |

TABLE 2
Treated animals dosed at 1 mg/kg at Day 7.
Fipronil dosed at 1 mg/kg

| | | | Day 7 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 89 | |
| 4882 | 2 | fipronil | 0 | 0 | 88 | |
| 4871 | 2 | fipronil | 3 | 0 | 25 | |
| 4864 | 2 | fipronil | 0 | 0 | 38 | |
| | | | | Group 2 | 50.3 | 43.4% |
| | | | | mean: | | |
| 4857 | 3 | untreated | 0 | 0 | 13 | |
| 4897 | 4 | fipronil | 0 | 0 | 12 | |
| 4881 | 4 | fipronil | 0 | 0 | 18 | |
| 4853 | 4 | fipronil | 0 | 0 | 1 | |
| | | | | Group 4 | 10.3 | 20.5% |
| | | | | mean: | | |

TABLE 3
Treated animals dosed at 2 mg/kg at Day 14.
Fipronil dosed at 2 mg/kg

| | | | Day 14 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 62 | |
| 4882 | 2 | fipronil | 0 | 0 | 1 | |
| 4871 | 2 | fipronil | 0 | 0 | 1 | |
| 4864 | 2 | fipronil | 0 | 0 | 0 | |
| | | | | Group 2 | 0.7 | 98.9% |
| | | | | mean: | | |
| 4857 | 3 | untreated | 0 | 0 | 10 | |
| 4897 | 4 | fipronil | 0 | 0 | 3 | |
| 4881 | 4 | fipronil | 0 | 0 | 3 | |
| 4853 | 4 | fipronil | 0 | 0 | 5 | |
| | | | | Group 4 | 3.7 | 63.3% |
| | | | | mean: | | |

TABLE 4
Treated animals dosed at 2 mg/kg at Day 21.
Fipronil dosed at 2 mg/kg

| | | | Day 21 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 64 | |
| 4882 | 2 | fipronil | 0 | 0 | 50 | |
| 4871 | 2 | fipronil | 0 | 0 | 8 | |
| 4864 | 2 | fipronil | 0 | 0 | 13 | |
| | | | | Group 2 | 23.7 | 63.0% |
| | | | | mean: | | |
| 4857 | 3 | untreated | 0 | 0 | 8 | |
| 4897 | 4 | fipronil | 0 | 0 | 13 | |
| 4881 | 4 | fipronil | 0 | 0 | 10 | |
| 4853 | 4 | fipronil | 0 | 0 | 15 | |
| | | | | Group 4 | 12.7 | −58.3% |
| | | | | mean: | | |

TABLE 5
Treated animals dosed at 4 mg/kg at Day 36.
Fipronil dosed at 4 mg/kg

| | | | Day 36 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 60 | |
| 4882 | 2 | fipronil | 0 | 0 | 0 | |
| 4871 | 2 | fipronil | 1 | 1 | 0 | |
| 4864 | 2 | fipronil | 0 | 0 | 0 | |
| | | | | Group 2 | 0.0 | 100.0% |
| | | | | mean: | | |
| 4857 | 3 | untreated | 0 | 0 | 8 | |
| 4897 | 4 | fipronil | 0 | 0 | 1 | |
| 4881 | 4 | fipronil | 0 | 0 | 2 | |
| 4853 | 4 | fipronil | 0 | 0 | 2 | |
| | | | | Group 4 | 1.7 | 79.2% |
| | | | | mean: | | |

TABLE 6
Treated animals dosed at 4 mg/kg at Day 43.
Fipronil dosed at 4 mg/kg

| | | | Day 43 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 55 | |
| 4882 | 2 | fipronil | 0 | 0 | 2 | |
| 4871 | 2 | fipronil | 0 | 1 | 1 | |
| 4864 | 2 | fipronil | 0 | 0 | 0 | |
| | | | | Group 2 | 1.0 | 98.2% |
| | | | | mean: | | |
| 4857 | 3 | untreated | 0 | 0 | 10 | |
| 4897 | 4 | fipronil | 0 | 0 | 6 | |
| 4881 | 4 | fipronil | 0 | 1 | 5 | |
| 4853 | 4 | fipronil | 0 | 0 | 1 | |
| | | | | Group 4 | 4.0 | 60.0% |
| | | | | mean: | | |

TABLE 7
Treated animals dosed at 4 mg/kg at Day 50.
Fipronil dosed at 4 mg/kg

| | | | Day 50 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 3 | 3 | 50 | |
| 4882 | 2 | fipronil | 0 | 0 | 58 | |
| 4871 | 2 | fipronil | 0 | 0 | 1 | |
| 4864 | 2 | fipronil | 0 | 0 | 0 | |
| | | | | Group 2 | 19.7 | 60.7% |
| | | | | mean: | | |

TABLE 7
Treated animals dosed at 4 mg/kg at Day 57.
Fipronil dosed at 4 mg/kg

| | | | Day 57 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 61 | |
| 4882 | 2 | fipronil | 0 | 0 | 22 | |
| 4871 | 2 | fipronil | 0 | 0 | 0 | |
| 4864 | 2 | fipronil | 0 | 0 | 1 | |
| | | | | Group 2 | 7.7 | 87.4% |
| | | | | mean: | | |

TABLE 8
Treated animals dosed at 4 mg/kg at Day 64.
Fipronil dosed at 4 mg/kg

| | | | Day 64 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 76 | |
| 4882 | 2 | fipronil | 0 | 0 | 62 | |
| 4871 | 2 | fipronil | 0 | 0 | 1 | |
| 4864 | 2 | fipronil | 0 | 0 | 0 | |
| | | | | Group 2 | 21.0 | 72.4% |
| | | | | mean: | | |

TABLE 9
Treated animals dosed at 4 mg/kg at Day 78.
Fipronil dosed at 4 mg/kg

| | | | Day 78 - | | Comb | |
|---|---|---|---|---|---|---|
| | | | Mortality Obs | | counts | % |
| Animal # | Group # | Treatment | 30 min | 1 hr | 24 hr | Efficacy |
| 4883 | 1 | untreated | 0 | 0 | 51 | |
| 4882 | 2 | fipronil | 0 | 0 | 14 | |
| 4871 | 2 | fipronil | 0 | 1 | 3 | |
| 4864 | 2 | fipronil | 0 | 0 | 3 | |
| | | | | Group 2 | 6.7 | 86.9% |
| | | | | mean: | | |

The invention having been fully described, those of ordinary skill in the art will recognize that it extends to equivalents and modifications thereof, without departing from the scope of the invention, which is defined by the appended claims.

1. A pharmaceutically acceptable composition comprising an ectoparasiticidally effective amount of fipronil for oral delivery to mammals to control external parasites over a period of at least 1 week and as long as 9 weeks.

2. The composition according to claim 1, wherein the ectoparasiticidally effective amount is sufficient to kill adult fleas or ticks.

3. The composition according to claim 1, wherein an additional active agent is provided to kill flea or tick eggs.

4. The composition according to claim 1, wherein fipronil is the only ectoparasiticidal agent present in the pharmaceutical composition.

5. The composition according to claim 4, wherein the fipronil is provided at a dose concentration between 1 mg/kg and 4 mg/kg.

6. A method for the control of external parasites on a mammal, the method comprising oral administration of an ectoparasiticidally effective amount of fipronil to the mammal to control ectoparasites thereon for a period of at least 1 week.

7. The method according to claim 6, wherein the external parasites are ticks or fleas.

8. The method according to claim 7, wherein at least 100% of fleas present on the treated mammal are killed within 24 hours of a single dose administration.

9. The method according to claim 7, wherein at least 79% of ticks present on the treated mammal are killed within 24 hours of a single dose administration.

10. The method according to claim 6, wherein the fipronil is the only ectoparasiticially effective agent administered.

11. The method according to claim 8, wherein the dose administered is between 1 mg/kg and 4 mg/kg.

12. The method according to claim 9, wherein the dose administered is between 1 mg/kg and 4 mg/kg.

13. The method according to claim 11, wherein the dose is administered no more often than once every 4 weeks.

14. The method according to claim 12, wherein the dose is administered no more often than once every 4 weeks.

15. The method according to claim 13, wherein the dose is administered no more often than once every 6 weeks.

16. The method according to claim 14, wherein the dose is administered no more often than once every 6 weeks.

17. The method according to claim 13, wherein the dose is administered no more often than once every 8 to 9 weeks.

18. The method according to claim 14, wherein the dose is administered no more often than once every 8 weeks.

19. The method according to claim 6, wherein an additional active agent is administered to kill flea or tick eggs.

20. The method according to claim 6, wherein an additional active agent is administered to provide killing of ectoparasites in less than 24 hours.
